(19) United States
(12) Patent Application Publication
(10) Pub. No.: US 2009/0298966 A1
(43) Pub. Date: Dec. 3, 2009

(54) DENTAL MATERIAL

(75) Inventors: Lorenzo Vanini, San Fedele Intelvi (IT); Eugenio Miceli, Genova (IT); Thomas Niem, Korbach (DE)

Correspondence Address: RANKIN, HILL & CLARK LLP, 38210 Glenn Avenue, WILLOUGHBY, OH, 44094-7808, US

(73) Assignees: GDF Gesellschaft Fur Dentale Forschung Und Innovationen GMBH, Rosbach v.d.H. (DE); Micerium SPA, San Fedele Intelvi (IT); Lorenzo Vanini, San Fedele Intelvi (IT)

(21) Appl. No.: 12/067,418

(22) PCT Filed: Sep. 22, 2005
(86) PCT No.: PCT/IB05/02816

§ 371 Date: Aug. 14, 2008

(51) Int. Cl. A61K 6/083; A61Q 11/00

(52) U.S. Cl. 523/116

(57) ABSTRACT

The dental material contains a polymerizable dental resin of index of refraction n1, inorganic nano-scale solid particles of index of refraction n2, dental glass particles of index of refraction n3, and accessory substances to autopolymerize and light-cure the dental resin. The dental material exhibits a composite index of refraction n4 in the range of 1.56 to 1.70. The dental material imparts optimal esthetics to the tooth being restored and allows for application in substantially thick layers.

The present invention relates to a dental material defined in the preamble of claim 1.

The known dental materials, in particular filling substances and dental enamels, are composed of the most diverse liquid and solid substances that in general have different indices of refraction. Moreover, in general the known dental materials' indices of refraction do not match those of natural teeth. As a result the optical effects. i.e. the esthetics of these dental materials are a drawback when they are applied in or on the teeth. Whereas the natural dental enamel becomes substantially whiter and more luminous as its thickness increases, hence its grayness decreases, the heretofore known dental materials behave indeed quite to the contrary in that their grayness increases with layer thickness (the glass effect). The esthetic properties of repairs/restorations attained using such known materials accordingly do not correspond to those of the natural enamel.

Accordingly there is demand for a dental material, in particular for reconstructing portions of enamel, which also allows applying it in thicker layers corresponding to the natural enamel on the tooth to be restored.

The objective of the present invention is to offer a remedy in the form of a dental material imparting optimally esthetic properties to the tooth being restored and also allowing being applied in thick layers.

The present invention solves this problem by a dental material comprising the features of claim 1.

Substantially the advantages offered by the present invention are the following:

(A) In particular when being used as a layer of enamel, the dental material of the present invention also may be deposited in layers of substantial thickness and thereby attains an esthetic optical effect of a natural dental enamel,

(B) The dental material of the present invention corresponds to the index of refraction of the natural tooth enamel and in particular it offers optimal transparency and depth effects,

(C) The possibility of depositing substantially thick layers of the material of the invention allows simpler and improved material handling,

(D) Said larger layer thickness also offers improved material resistance to the chemical actions of the mouth ambience.

In a particular embodiment mode of the present invention, the dental material's index of refraction n₄ larger than 1.59, preferably larger than 1.60. The dental material's index of refraction n₄ of the dental material advantageously shall be smaller than 1.64, preferably less than 1.62.

In another embodiment mode, the dental material which was exclusively mixed with nano-size (hereafter nano) inorganic solid particles exhibits an index of refraction n₁₂ deviating at most by 5%, preferably 2% from dental glass particle's index of refraction n₃. The admixed, inorganic nano-particles admixed in a first step into the dental resin result in a clear and transparent, i.e. anon-opaque, colloidal system. When selecting a dental glass having an approximately identical index of refraction, the overall system's optical properties are substantially retained, in other words, the liquid or pasty dental material also remains transparent on the whole, a feature of large importance in dental applications.

Advantageously the index of refraction n₁₂ shall deviate no more than 1.0%, preferably no more than 0.2% from the index of refraction n₃ and preferably shall be within the range of 1.56 through 1.65, preferably from 1.59 through 1.62.

Advantageously the dental glass particles' index of refraction n₃ shall be in the range from 1.58 to 1.65, preferably from 1.59 to 1.62. The dental resin's index of refraction n1 advantageously shall be larger than 1.54.

Bis-methacrylates, preferably bis-GMA or an ethoxylated bisphenol-A-dimethylacrylates or derivatives thereof were found suitable as dental resins of the present invention. Such a dental resin also may be in the form of a methacrylate-substituted poly-di-phenyl silicone or a diphenylsilane derivative. The following substances are also suitable: dental resins from the group of poly(pentabromophenyl)-methacrylates, poly(pentabromophenyl-acrylates), poly(pentabromobenzyl-methacrylates), poly(penta-bromobenzyl-acrylates), poly(2,4,6-tribromophenyl-methacrylates), poly(vinylphenylsulfides), poly(1-naphthyl-methacrylates), poly(2-vinylthiophenes), poly(2,6-dichlorostyrenes), poly(n-vinylphthalimides), poly(2-chlorostyrenes), poly(pentachlorophenyl methacrylates).

The nano-scale inorganic solid particles advantageously are selected being based on metal oxides, preferably titanium dioxide, zinc oxide, zirconium oxide or aluminum oxide as well as mixtures thereof. In general particles of diameters less than 100 nm and preferably between 20 and 40 nm shall be used.

In a preferred embodiment mode of the present invention, the ratio by weight of the dental resin to the nano-scale inorganic solid particles is in the range of 1.0 to 2.5.

The surface of the nano-scale inorganic solid particles may be coated with an organic acid, preferably a carboxylic acid derivative or a methacrylate-substituted silane. Such a coating precludes the nano-scale inorganic solid particles from re-agglomerating in the dental resin. Surprisingly this useful effect is preserved even after adding the dental glass particles to the colloidal dental resin/nano particles system. 2-[2-(2-methoxyethoxy)-ethoxy]acetic acid or gamma-methacryloxypropyltrimethoxy silane are especially suitable.

Preferably the dental glass particle shall exhibit a maximum mean diameter of 5μ, preferably no more than 0.7μ.

Especially appropriate dental glasses are: borosilicate glasses, barium-aluminosilicate glasses, silica, titanium silicate, zirconium silicate, barium-magnesium-aluminosilicate glasses, barium oxide, quartz and aluminum oxide. Furthermore the dental glasses may also be gamma-methacryloxypropyltrimethoxysilane-modified.

Appropriate dental glasses are amorphous, spherical materials based on mixed oxides of SiO₂, ZrO₂, ZnO, La₂O₃, Al₂O₃ and/or TiO₂ having a mean, average particle size of 0.005 to 5.0 μm, preferably between 0.1 and 1 μm, as well as macro and mini fillers such as powders of quartz, glass ceramics or glass, barium silicate glasses, barium fluorosilicate glasses and Li/Al silicate glasses, barium glasses, the oxides of aluminum, zinc, lanthanum, zirconium or silicon having an average particle size of 0.01 to 20 μm, preferably 0.5 to 5 μm. The expressions mini-fillers and maxi-fillers respectively denote fillers or particle sizes between 0.5 and 1.5 μm and fillers of particle sizes between 10 and 20 μm.

The following are further appropriate dental glasses: Mixtures of (A), namely amorphous, spherical particles of silicon dioxide and up to 20 molar % of an oxide of at least one element of groups 1, II, IV, V, XII, XIII and XIV of the periodic table having an index of refraction of 1.50 to 3.00 and an average primary particle size of 0.1 to 5.0 μm, and (B), namely powders of quartz, glass ceramics or glass or their mixtures having an index of refraction of 1.50 to 2.00 and an average particle size of 0.1 to 5.0 μm.

Moreover the components (A) and (B) may be used per se as dental glass components.

The oxide of the inorganic filler (A) is an oxide of a metal from the groups I, II, IV, V, XII, XIII and XIV of the periodic table, preferably the oxide of strontium, aluminum, zinc, titanium, lanthanum and/or zirconium. Preferably the average primary particle size is in the range from 0.15 to 2.0μ and the index of refraction of the inorganic filler (A) preferably shall be between 1.57 and 1.64. An especially preferred value of this index of refraction is 1.60±0.02. The type (A) filler also may be used in sintered form as a mixture of agglomerates with an average particle size of 0.5 to 2.0 μm.

Preferably the average primary particle size of the inorganic filler (B) is between 0.1 and 5.0 μm and in especially preferred manner it is between 0.5 and 2.0 μm, the index of refraction preferably being between 1.57 and 1.64. Mixtures of fillers may also be used. The invention preferably uses Ba silicate glasses having a mean grain size in the range of 0.4 to 2.0 μm and Li/Al silicate glasses having a mean grain size of 0.4 to 2.0 μm.

The surface of the dental particles may be fitted with a crosslinking adhesive comprising double bonds, preferably gamma-methacryloxypropyltrimethoxy silane.

Advantageously the dental glass particles contain less than 35% by weight (wt) of silicon oxide, advantageously less than 30% by wt. This feature offers the advantage over glasses of higher silicon oxide proportions that more ZrO₂ or ZnO etc. may be added, the latter increasing the index of refraction.

The nano-scale inorganic solid particles, preferably zirconium oxide, advantageously offer a unit surface mass in the range of 110-250 m2/g.

The unit surface per mass of the dental glass particles advantageously are within the range of 12-15 m²/g (for 0.7μ particle size), 7-8 m²/g (for 1.0μ particle size) and 1-2 m²/g (for 5.0μ particle size).

Advantageously the dental glass particle's density is in the range of 2.5-4.7 g/cm³ (in particular 3.42 g/cm³). The nano-scale inorganic solid particles preferably shall have a density of 3.0-6.5 g/cm³ (in particular 5.4 g/cm³).

The index of refraction of the hardened/cured dental material advantageous is within the range of 1.56-1.70, preferably the range of 1.58-1.64.

The present invention and further embodiment modes of it are elucidated below in relation to several modes of implementation.

### EXAMPLE 1

### Preparing a Light-Curing Dental Resin Mixture

A dental resin mixture composed of the following components was prepared in a conventional planetary mixer. The resulting homogeneous mixture's index of refraction n_{D}²⁰=1.5140:

30.0 g urethane-dimethacrylate,

56.0 g bis-GMA

15.0 g hexanedioldimethacrylate,

0.20 g camphor quinone

0.20 g ethyl-p-dimethylaminobenzoate.

### EXAMPLE 2

### Preparing a Dental Resin Mixture Containing Zirconium Oxide

160.0 g sol containing zirconium oxide in ethanol (40%) are admixed to the 50.0 g of the dental resin mixture prepared in Example 1. The size of the zirconium oxide particles was 4-20 nm. For that purpose the dental resin mixture was completely dissolved in the sol by agitating with a magnetic stirrer or by shaking. The resulting dental resin mixture containing zirconium oxide was rid of easily volatile components using a rotary evaporator and under darkened conditions and was dried 30 min. at room temperature in vacuum. The index of refraction of the mixture so obtained was n_{D}²⁰=1.5880.

### EXAMPLE 3

### Preparing a Mixture of Dental Resins Containing Zirconium Oxide

180 g of a sol containing zirconium oxide in ethanol (40%) were admixed similarly to Example 2 to 50.0 g of the dental resin mixture prepared in Example 1. The resulting dental resin mixture containing zirconium oxide was rid ofs easily volatile components using a rotary evaporator and under darkened conditions and dried at room temperature in vacuum for 30 min. The index of refraction of the mixture so prepared was n_{D}²⁰=1.6050.

### EXAMPLE 4

### Preparing a Dental Composite

130.0 g of silanized glass powder were admixed to 85.0 g of the mixture prepared in Example 3. The glass powder properties wee as follows:

Mean particle size: 0.7μ,
Index of refraction n_{D}²⁰: 1.6000

### Density (ρ): 3.42

### Composition: 30% SiO₂/25% SrO/10% ZnO/10% ZrO₂/

Remainder: B₂O₃/Al₂O₃/La₂O₃/CaO/Na₂O.

This mixture was processed in a conventional planetary mixer into a homogeneous paste of an index of refraction n_{D}²⁰=1.6060.

A sample of the paste was cured for 9 minutes using a conventional light polymerizer (Spektra 2000; Schütz-Dental). The properties of cured composite so made were as follows:

Index of refraction n_{D}²⁰=1.6060
Vickers surface hardness 44.5
Vickers hardness at 2 mm depth: 15.9

### EXAMPLE 5

### Preparing a Dental Composite

130.0 g of silanized glass powder were admixed to 85.0 g of the mixture of Example 3. The properties of the glass powder were as follows.

Mean particle size: 1.0 μm
Index of refraction n_{D}²⁰=1.6000

### Density (ρ) 3.42

### Composition 30% SiO₂/25% SrO/10% ZnO/10% ZrO₂/

Remainder: B₂O₃/Al₂O₃/La₂O₃/CaO/Na₂O.

This mixture was processed in a conventional planetary mixer into a homogeneous paste and exhibited an index of refraction n_{D}²⁰=1.6050. A paste sample was cured for 9 minutes using a conventional light polymerizer (Spektrum 2000; Schütz-Dental). The properties of the composite so made were the following:

Index of refraction n_{D}²⁰=1.6050
Vickers surface hardness 49.9
2 mm depth Vickers hardness: 34.1

### EXAMPLE 6

### Preparing a Dental Resin Mixture

A dental mixture of the components below was prepared in a conventional planetary mixer. The index of refraction of the final mixture was n_{D}²⁰=1.4960:

40 g bis-GMA
88 g urethane dimethacrylate
32 g hexanedioldimethacrylate
0.32 g camphor quinine
0.32 g ethyl-p-dimethylaminobenzoate.

### EXAMPLES 7-16

### Preparing the Dental Resin Mixtures Containing Zirconium Oxide

The Table below shows the index of refraction of the dental resin mixture of Example 6 and a sol containing zirconium oxide as a function of content of zirconium oxide.

| | Content in g of | Content in g of | Index of |
|---|---|---|---|
| | monomer mixture of | Zirconium oxide | refraction |
| Example # | Example 6 | sol 40% | n_{D}²⁰ |
| 7 | 160 | 225 | 1.5440 |
| 8 | 160 | 320 | 1.5590 |
| 9 | 160 | 384 | 1.5670 |
| 10 | 160 | 417 | 1.5710 |
| 11 | 160 | 448 | 1.5825 |
| 12 | 160 | 480 | 1.5860 |
| 13 | 160 | 512 | 1.5950 |
| 14 | 160 | 622 | 1.6025 |
| 15 | 160 | 800 | 1.6240 |
| 16 | 160 | 874 | 1.6350 |

### EXAMPLE 17

### Preparing a Dental Composite

130.0 g of silanized glass powder were admixed to 85.0 g of the mixture prepared in Example 13. The glass powder properties were as follows:

particle size 1.0μ
index of refraction n_{D}²⁰=1.6000
density (ρ) 3.42
composition 30% SiO₂/25 SrO/10 ZnO/5% La₂O₃/5% Al₂O₃/

remainder B₂O₃/CaO/Na₂O/ZrO₂.

This mixture was processed in a conventional planetary mixer into a homogeneous paste. The paste sample was cured using a conventional light polymerizer (Schütz-Dental) for 9 minutes to completion. The properties of the cured composite were as follows:

n_{D}²⁰=1.5955
Vickers surface hardness 52.7
2-mm deep Vickers hardness 40.1.

### EXAMPLE 18

### Preparing a Dental Composite

130.0 g of silanized glass powder were admixed to 85.0 g of the mixture made in Example 13. The glass powder properties were as follows:

mean particle size 0.7 μm
index of refraction n_{D}²⁰=1.6000
density (ρ) 3.42
composition 30% SiO₂/25% SrO/10% Zno/10% ZrO₂/

remainder B₂O₃/Al₂O/La₂O₃/CaO/Na₂O.

This mixture was processed in a conventional planetary mixer into a homogeneous paste. A paste sample was cured for 9 minutes using a conventional light polymerizer (Spektra 2000; Schütz-Dental). The properties of the finished, cured composite were the following:

n_{D}²⁰=1.5945
Vickers surface hardness 50.5
2 mm deep Vickers hardness 35.5.

### EXAMPLE 19

### Preparing a Dental Resin Mixture Containing Zirconium Oxide

A dental resin mixture of the components below was prepared in a conventional planetary mixer (similar to that of Example 2). The index of refraction of the final homogeneous mixture was n_{D}²⁰=1.5920.

50.0 g bis-GMA (index of refraction n_{D}²⁰=1.5503
100.0 g 40% zirconium-oxide containing sol in ethanol
0.05 g camphor quinone
0.05 g ethyl-p-dimethylaminobenzoate.

### EXAMPLE 20

### Preparing a Dental Composite

100 g of the mixture made in Example 19 were mixed with 130 g of silanized glass powder. The properties of the glass powder were as follows:

mean particle size 0.7 μm
index of refraction n_{D}²⁰=1.6000
density (ρ) 3.42
composition 30% SiO₂/25% SrO/10% ZnO/10% ZrO₂/

remainder B₂O₃/Al₂O₃/La₂O₃/CaO/Na₂O.

This mixture was processed in a conventional planetary mixer into a homogeneous paste. The paste sample was cured using a conventional light polymerizer (Spektra 2000; Schütz-Dental) for 9 minutes. The properties of the final, cured composite were as follows:

n_{D}²⁰=1.6010
Vickers surface hardness 35.8
2-mm deep Vickers hardness 19.5

### EXAMPLE 21

### Preparing a Dental Composite

100 g of the mixture made in Example 19 were mixed with 130 g of silanized glass powder. The glass powder properties were as follows:

mean particle size 5.0μ
index of refraction n_{D}²⁰=1.6000
density (ρ) 3.42
composition 30% SiO₂/25% SrO/10% ZnO/5% La₂O₃/5% Al₂O₃/

remainder: B₂O₃/CaO/Na₂O/ZrO₂

This mixture was processed in a conventional planetary mixer into a homogeneous paste. A paste sample was cured for 9 minutes in a conventional light polymerizer (Spektra 2000; Schütz-Dental). The properties of the cured composite so made were as follows:

n_{D}²⁰=1.6060
Vickers surface hardness 27.4
2-mm deep Vickers hardness 18.2.

### EXAMPLE 22

### Preparing an Autopolymerizable 2-Component Dental Resin Mixture

Fundamental Mixture:

30.0 g urethane-dimethacrylate
55.0 g bis-GMA
15.0 g hexanedioldimethacrylate,

Dental Resin Mixture Containing Zirconium Oxide:

50.0 g of above fundamental resin mixture
180.0 g a sol containing zirconium oxide in ethanol (40%),

Component A:

85.0 g of the above dental resin mixture containing zirconium oxide
0.27 g of N,N-bis-(2)-hydroxyethyl)-p-toluidine
130.0 g glass powder (mean particle size: 0.7μ),

Component B:

85.0 g of the above dental resin mixture containing zirconium oxide
12.5 g dibenzoylperoxide (50% powder in phthalate)
130.0 g glass powder (0.7μ).

Equal amounts of components A and B were mixed using a spatula for 30 seconds and this quantity was used to restore an enamel lesion. The cured material on the tooth offered high translucency and an appearance matching well that of natural enamel.

The dental material of the invention may be prepared in both flowable and kneadable form. At a layer thickness of 1 mm, all cured dental composites offered excellent transparency.

1. A dental material containing a polymerizable dental resin exhibiting an index of refraction n₁ and comprising nano-scale inorganic solid particles exhibiting the index of refraction n₂, further glass particles having the index of refraction n₃, and accessory substances to autopolymerize or light-cure the dental resin
A) the dental material exhibits a compound index of refraction n₄ in the range of 1.56-1.70; and
B) the index of refraction n₅ of the cured dental material is in the range of 1.56 to 1.70.

2. Dental material as claimed in claim 1, wherein the index of refraction n₄ is larger than, 1.59.

3. Dental material as claimed in claim 1, wherein the index of refraction n₄ is less than 1.64.

4. Dental material as claimed in claim 1, wherein the dental resin mixed solely with the inorganic nano-scale solid particles exhibits an index of refraction n₁₂ which at most is higher than 5, preferably at most higher by 2% than the dental glass particles' index of refraction n₃.

5. Dental material as claimed in claim 4, wherein the index of refraction n₁₂ at most is higher by 1.0% than the index of refraction n₃.

6. Dental material as claimed in claim 4, wherein the index of refraction n₁₂ is in the range of 1.56 through 1.65.

7. Dental material as claimed in claim 4, wherein the index of refraction n₃ is in the range of 1.58 through 1.65.

8. Dental material as claimed in claim 1, wherein the dental resin's index of refraction n₁ is larger than 1.54.

9. Dental material as claimed in claim 1, wherein the dental resin is a bis-methacrylate or an ethoxylated bisphenol-A-dimethacrylate or derivatives thereof.

10. Dental material as claimed in claim 1, wherein the dental resin is a methacryl-substituted poly-di-phenyl-silicone or diphenyl silane derivative.

11. Dental material as claimed in claim 1, wherein the dental resin is selected from the group of poly(pentabromophenyl-methacrylates), poly(pentabromophenyl-acrylates), poly(pentabromobenzyl-methacrylates), poly(penta-bromobenzyl-acrylates, poly(2,4,6-tribromophenyl-methacrylates), poly(vinylphenyl sulfides), poly(1-naphthylmethacrylates), poly(2-vinylthiophenes, poly(2,6-di-chlorostyrenes), poly(n-vinylphthalimides), poly(2-chlorostyrenes), poly(pentachlorophenylmethacrylates).

12. Dental material as claimed in claim 1, wherein the inorganic nano-scale solid particles are selected to be metal oxides.

13. Dental material as claimed in claim 1, wherein the ratio by weight of dental resin to the inorganic nano-scale solid particles is in the range of 1.0 to 2.5.

14. Dental material as claimed in claim 1, wherein the surface of the inorganic nano-scale solid particles is coated with an organic acid, preferably a carboxylic acid or a methacrylate-substituted silane.

15. Dental material as claimed in claim 1, wherein the dental glass particles' mean maximum diameter is 5 μm, preferably at most 0.7 μm.

16. Dental material as claimed in claim 1, wherein the dental glass particles' surface is fitted with an adhesive means comprising crosslinking double bonds, preferably gamma-methacryloxypropyltrimethoxysilane.

17. Dental material as claimed in claim 1, wherein the dental glass particles contain less than 35% by wt of silicon oxide.

18. Dental material as claimed in claim 12, wherein the inorganic nano-scale solid particles exhibit a unit surface mass in the range of 110-250 m²/g.

19. Dental material as claimed in claim 1, wherein the dental glass particles' unit surface mass is in the range of 12-15 m²/g (for 0.7μ particle size), 7-8 m²/g (for 1.0μ particle size) and 1-2 m²/g (for 5.0μ particle size).

20. Dental material as claimed in claim 1, wherein the dental glass particles' density is between 2.5 and 4.7 g/cm³.

21. Dental material as claimed in claim 1, wherein the inorganic nano-scale solid particles' density is between 3.0 and 6.5 g/cm³.

22. Dental material as claimed in claim 1, wherein the cured dental material's index of refraction n₅ is in the range of 1.56 to 1.70.

23. Dental material as claimed in claim 1, wherein the inorganic nano-scale particles have a diameter of less than 100 nonometers.

24. Dental material as claimed in claim 1, wherein after having cured it offers a high transparency and preferably at a layer thickness of 1 mm, an excellent transparency.
